(19) United States
(12) Patent Application Publication
(10) Pub. No.: US 2012/0078140 A1
(43) Pub. Date: Mar. 29, 2012

(54) Method and Apparatus for Removing Blood Clots and Tissue from the Patient's Head

(75) Inventor: Henry Nita, Alameda, CA (US)

(73) Assignee: Penumbra, Inc., Alameda, CA (US)

(21) Appl. No.: 13/312,624

(22) Filed: Dec. 6, 2011

Related U.S. Application Data
(63) Continuation-in-part of application No. 13/199,557, filed on Sep. 2, 2011.
Application No. 13/199,557 is a continuation-in-part of application No. 13/136,075, filed on Jul. 22, 2011.
Application No. 13/136,075 is a continuation-in-part of application No. 12/930,364, filed on Jan. 4, 2011.
Application No. 12/930,364 is a continuation-in-part of application No. 12/799,706, filed on Apr. 30, 2010.
Application No. 12/799,706 is a continuation of application No. 11/203,738, filed on Aug. 15, 2005, now Pat. No. 7,717,853.
Application No. 11/203,738 is a continuation-in-part of application No. 11/165,872, filed on Jun. 24, 2005, now abandoned.

(51) Int. Cl. A61N 7/00

(52) U.S. Cl. 601/2

(57) ABSTRACT

Methods and apparatus for removing blood clots and tissue from intracranial space of the patient's head using an ultrasound device under visualization guidance to locate, dissolve and remove the blood clots outside the patient's head are provided. One or more pharmacologic agents may be delivered to the treatment area to further facilitate removal of the blood clots or other tissue.

### RELATED CASES

This is a continuation-in-part of co-pending application Ser. No. 13/199,557 filed on Sep. 2, 2011 which is a continuation-in-part of co-pending application Ser. No. 13/136,075, filed on Jul. 22, 2011, which is a continuation-in-part of co-pending application Ser. No. 12/930,364, filed on Jan. 4, 2011, which is continuation-in-part of co-pending application Ser. No. 12/799,706, filed on Apr. 4, 2010, which is continuation of Ser. No. 11/203,738, filed Aug. 15, 2005, now U.S. Pat. No. 7,717,853, which is a continuation-in-part of Ser. No. 11/165,872, filed Jun. 24, 2005, now abandoned, whose disclosures are incorporated by this reference as though fully set forth herein.

### BACKGROUND OF THE INVENTION

1. Field of the Invention

The present invention relates generally to medical methods and apparatus. More specifically, the invention relates to methods and apparatus for intracranial ultrasound delivery for the treatment of blood clots, and for dissolving blockages inside and outside intracranial aneurysms using ultrasound energy.

Stroke is characterized by the sudden loss of circulation to an area of the brain, resulting in a corresponding loss of neurologic function. Also called cerebrovascular accident or stroke syndrome, stroke is a nonspecific term encompassing a heterogeneous group of pathophysiologic causes, including thrombosis, embolism, and hemorrhage. Strokes currently are classified as either hemorrhagic or ischemic. Hemorrhagic stroke is bleeding that occurs inside the skull, a serious medical emergency that crushes delicate brain tissue, limits its blood supply and causes potentially deadly brain herniation in which parts of the brain are squeezed past structures in the skull. Blood irritates the brain tissues, causing swelling, and collects into a blood clot mass called a hematoma. Either swelling or a hematoma will increase pressure on brain tissues and can rapidly destroy them. Acute ischemic stroke refers to strokes caused by thrombosis or embolism and accounts for 80% of all strokes, and the other 20% are caused by hemorrhagic stroke and blood clot formation in intracranial space.

More than 400,000 people per year in the U.S. have a first-time stroke. At current trends, this number is projected to increase to one million per year by the year 2050. Stroke is the third leading cause of death and the leading cause of disability in the U.S. Worldwide, cerebrovascular disease was the second leading cause of death in 1990, killing over 4.3 million people. Cerebrovascular disease was also the fifth leading cause of lost productivity, as measured by disability-adjusted life years (DALYs). In 1990, cerebrovascular disease caused 38.5 million DALYs throughout the world. And although stroke often is considered a disease of the elderly, 25% of strokes occur in persons younger than 65 years. When the direct costs (care and treatment) and the indirect costs (lost productivity) of strokes are considered together, strokes cost US society today over $50 billion per year.

Until very recently, almost nothing could be done to help patients with acute stroke. Little treatment existed for ischemic stroke until 1995, when the National Institute of Neurologic Disorders and Stroke (NINDS) recombinant tissue-type plasminogen activator (rt-PA) stroke study group first reported that the early administration of rt-PA benefited some carefully selected patients with acute ischemic stroke. Encouraged by this breakthrough study and the subsequent approval of t-PA for use in acute ischemic stroke by the U.S. Food and Drug Administration, administration of t-PA has become increasingly more prevalent in stroke treatment. Treating patients early enough in the course of stroke, however, is an extremely challenging hurdle to effective treatment of stroke. Furthermore, t-PA for stroke treatment is much more effective if delivered locally at the site of blood vessel blockage, but such delivery requires a great deal of skill and training, which only a small handful of medical professionals possess.

One proposed enhancement for treatment of stroke is the administration of trans-cranial Doppler (TCD) at high frequencies (i.e., approximately 2 MHz) and low intensities, which is normally used for diagnostic functions. TCD has been shown not only to be effective in visualizing clots, but also to be effective in lysing clots in the middle cerebral arteries, in combination with lytic drugs such as t-PA and/or microbubbles. TCD has also been shown to be safe, with no clinically significant brain bleeding effects. (See, for example: A. V. Alexandrov et al., “Ultrasound-Enhanced Thrombolysis for Acute Ischemic Stroke,” N. Engl. J. Med. 351; 21, Nov. 18, 2004; and W. C. Culp and T. C McCowan, “Ultrasound Augmented Thrombolysis,” Current Medical Imaging Reviews, 2005, 1, 5-12.) The primary challenge in using TCD to enhance stroke treatment, however, is that the skull attenuates the ultrasound signal to such a high degree that it is very difficult to deliver high-frequency, low-intensity signals through the skull. Using higher intensity ultrasound signals, in an attempt to better penetrate the skull, often causes unwanted bleeding of small intracranial blood vessels and/or heating and sometimes burning of the scalp. The only other option is to carefully aim a high-frequency, low-intensity TCD signal through a small window in the temporal bone of the skull to arrive at the middle cerebral artery, which is the technique described in the studies cited above and is the only technique studied thus far.

There are two main drawbacks to delivering high-frequency TCD through the temporal window. First, such delivery requires a high level of skill, and only a small handful of highly trained ultrasonographers are currently capable of performing this technique. Second, not all intracranial blood vessels are reachable with TCD via the temporal window. For example, although the temporal window approach may work well for addressing the middle cerebral artery, it may not work as well for reaching the anterior cerebral artery or various posterior intracranial arteries.

Assuming effective ultrasound delivery is achieved, in addition to enhancing treatment of acute thrombotic or embolic ischemic stroke, TCD may also enhance and/or facilitate treatment of other cerebral disorders. For example, recurrent lacunar strokes, dementia, head trauma patients with intracerebral blood clots or perfusion abnormalities, and even Alzheimer's patients may benefit from TCD. In any such disorders, administration of TCD may help restore normal blood flow to the brain, help disperse harmful blood clots inside or outside blood vessels, and/or cause hyper-perfusion in one or more areas of the brain, thus enhancing cerebral function. For example, ultrasound administration has been shown to enhance the production of nitric oxide in or nearby blood vessels, which may thus cause vasodilation of nearby arteries and arterioles and enhance tissue perfusion. (See, for example, W. Steffen et al., “Catheter-Delivered High Intensity, Low Frequency Ultrasound Induces Vasodilation in Vivo,” European Heart Journal (1994) 15, 369-376.) In any such treatments, however, use of TCD faces the same challenges i.e., it is very difficult to deliver at safe and effective frequencies to desired locations in the brain and thus can be performed only by a small handful of highly skilled technicians and can be directed only to a few areas in the brain. Also, the high intensities required to transmit ultrasound through the skull in TCD make its utility for treating any chronic disorder impractical, since any implantable power source used with a chronic, implantable ultrasound delivery device would be depleted too quickly.

Therefore, it would be desirable to have improved methods and apparatus for intracranial delivery of ultrasound energy for diagnostic ultrasound, therapeutic ultrasound, or both. Ideally, such techniques would be usable by a larger number of medical professionals than are currently qualified to administer TCD. Also ideally, such techniques would use ultrasound frequencies that do not cause unwanted bleeding in other blood vessels in the brain and that do not cause overheating or burning of the skin, while dissolving clots inside or outside the intracranial vessel(s). At least some of these objectives will be met by the present invention.

An intracranial hematoma occurs when a blood vessel ruptures within the brain or between the skull and the brain. Removing or reducing hematoma in the brain is crucial to the patient's recovery. Catheter-based evacuation is a novel surgical approach for the treatment of brain hematoma. Such a minimally invasive treatment of intracranial hematoma may help prevent complications and promote illness recovery, reduce morbidity and improve cure rate, and reduce medical costs. Removal of hematoma is performed using extraventicular drains (EVD) or drainage catheters. These drainage catheters or introducers placed inside hematoma not only provide a path for the brain to keep it decompressed, but also provide a channel to remove or reduce hematoma outside the patient's head. If drainage catheters or introducers become occluded, clogged, or obstructed, as it often does with fibrinous or blood clots, a permanent brain damage can occur. In such a case, the patient needs to return for placement of a new drainage catheter, which is a time consuming and cumbersome surgical procedure.

EVD catheters are different from shunts. Unlike drainage catheters, shunts allow excess cerebrospinal fluid to drain to another area of the body. Hydrocephalus, also known as “water on the brain,” is a medical condition in which there is an abnormal accumulation of cerebrospinal fluid (CSF) in the ventricles, or cavities of the brain. This may cause increased intracranial pressure inside the skull and progressive enlargement of the head, convulsion, tunnel vision, and mental disability or death. Typically, the fluid gets “shunted” (moved) into other body cavities, from where it can be reabsorbed.

Therefore, it would be desirable to have improved methods and apparatus for preventing blockages caused by EVD catheters or introducers, and/or recanalizing them without a need for additional replacement surgery.

Aneurysms in the brain occur when there is a weakened area in the wall of a blood vessel. Cerebral aneurysms involve widening of an entire blood vessel, or a “ballooning out” of part of a blood vessel, and can occur in any blood vessel that supplies the brain. Atherosclerosis, trauma, and infection, which can injure the blood vessel wall, can cause cerebral aneurysms.

A cerebral aneurysm may begin to “leak” and cause a severe headache. This could be a warning sign of a potential rupture that is dangerous and often catastrophic. Three common methods are used to repair an aneurysm: (i) clipping is the most common way to repair an aneurysm that requires open brain surgery (craniotomy); and endovascular repair—(a) using a “coil” or coiling, is a less invasive way to treat some aneurysms; and (b) using a minimally invasive implant structure deployed inside the blood vessel at the aneurysm or aneurysm neck to divert blood away from an aneurysm.

Cerebral aneurysms are classified both by size and shape. Small aneurysms have a diameter of less than 5 mm and rarely require treatment, medium (5 to 15 mm), large (15-25 mm), giant (25 to 50 mm), and super giant (over 50 mm).

There are three types of cerebral aneurysm. A saccular aneurysm is a rounded or pouch-like sac of blood that is attached by a neck or stein to an artery or a branch of a blood vessel. Also known as a berry aneurysm (because it resembles a berry hanging from a vine), this most common form of cerebral aneurysm is typically found on arteries at the base of the brain. Saccular aneurysms occur most often in adults. A lateral aneurysm appears as a bulge on one wall of the blood vessel, while a fusiform aneurysm is formed by the widening along all walls of the vessel.

A common location of cerebral aneurysms is on the arteries at the base of the brain, known as the Circle of Willis. Approximately 85% of cerebral aneurysms develop in the anterior part of the Circle of Willis, and involve the internal carotid arteries and their major branches that supply the anterior and middle sections of the brain. The most common sites include the anterior cerebral artery and anterior communicating artery (30-35%), the bifurcation, division of two branches, of the internal carotid and posterior communicating artery (30-35%), the bifurcation of the middle cerebral artery (20%), the bifurcation of the basilar artery, and the remaining posterior circulation arteries (5%).

The use of blood diverting devices such as balloon expanding or self expending stents that are preventing or limiting blood flow from intracranial arteries into to the aneurysm has been gaining a lot of clinical popularity and are often used for medium and larger aneurysms. Such aneurysm therapy does not require deployment of coils to create clots, rather it insulates an aneurysm from vascular blood flow and blood clots are created on their own. However, over time, some of aneurysms treated with such diverters have a tendency to grow or re-grow which could trigger a “mass effect”. Such events may compress or irritate surrounding brain tissue and structures, causing symptoms such as continuous morning headaches, nausea, loss of function in one or more of one of the nerve bundles in the brain or spinal cord leading to facial muscle weakness, double vision, impaired balance or hearing, tongue deviation, and weakness in the limbs, etc.). A shortcoming of these blood diverting devices is that it is impossible to place a micro-catheter through them to deploy coils and stop the aneurysm re-growth. Also, diverting blood flow implants cannot be and removed from the blood vessels and replaced. The only therapy to address such clinical problems is surgery/craniotomy to remove these aneurysms.

Therefore, there remains a need for improved methods and apparatus for removing blood clots from intracranial aneurysms using less invasive methods utilizing burr hole(s) or twist hole(s), and devices that provide the ability to remove them without a need for craniotomy.

Removing hematoma using EVDs usually takes from a few hours to several days or even weeks. While EVDs help to quickly decrease brain pressure and slowly reduce potentially deadly brain herniation, blood clots continue to irritate the brain tissue, causing swelling until removed outside the patient's head. Another approach to remove hematoma from the patient's intracranial space is craniotomy. It is a very complex neurosurgery that requires a team of several people and it is difficult or impossible to be done on emergency basis (i.e., within minutes or an hour from the time when patient arrives to the hospital). During the craniotomy, neurosurgeons open a significant portion of the skull to get access to the hematoma and to directly visualize and distinguish blood clots from brain tissue to be able to safely remove the blood clots. Craniotomies are high risk surgeries with a long recovery time.

Therefore, there is a need for improved methods and tools to remove intracranial hematoma using less invasive methods utilizing burr hole(s), devices that provide appropriate visualization of the blood clots and ability to remove them without a need for craniotomy, wherein the blood clots are freely residing within patient's intracranial space or inside aneurismal areas in the patient's head.

2. Background Art

U.S. Pat. No. RE36,939, issued to Tachibana et al., describes the use of microbubbles to enhance the effects of ultrasound delivery, with or without a pharmacological composition. U.S. Pat. No. 4,698,058, issued to Greenfield et al., discloses ultrasonic self cleaning catheter system for indwelling drains and medication supply. U.S. Pat. Nos. 5,929,901; 6,275,255; 6,310,642, issued to Adair et al., describe imaging devices incorporated within surgical instruments. U.S. Pat. No. 6,006,123, issued to Li et al., discloses use of ultrasound energy to enhance bioavailability of pharmaceutical agents. U.S. Pat. No. 5,399,158, issued to Lauer et al., describes a method of lysing thrombi, involving administration of t-PA or other plasminogen activators, with pulsed mode ultrasound. U.S. Pat. No. 6,368,330, issued to Hynes et al., is directed to an apparatus for frameless stereotactic surgery. Pub. No.: 2008/0319376 (Wilcox) describes method and apparatus for intracranial hemorrhages. Pub. No.: 2008/015181 (Khanna) describes nervous central system ultrasonic drain. Pub. No.: 2007/0005121 (Khanna) describes central nervous system cooling catheter with ultrasonic component. Pub. No.: 2011/01666592 (Garcia et al.) describes an implantable device to treat vascular malformations such as aneurysm. Pub. No.:2006/0206201 (Garcia et al.) describes an implantable device to embolize and occlude cerebral aneurysm. U.S. Pat. No. 6,533,722 issued to Nakashima describes electronic endoscopic camera having reduced diameter.

### BRIEF SUMMARY OF THE INVENTION

In one aspect of the present invention, a method for removing blood clots from an aneurysm in a patient's head using ultrasound energy includes forming at least one aperture in the patient's skull, advancing an ultrasound device into the aneurysm, and transmitting ultrasound energy from the ultrasound delivery device. In some embodiments, one hole is placed in the skull, and one ultrasound delivery device is used. In alternative embodiments, multiple holes are formed in the skull, and at least one ultrasound delivery device is advanced at least partway through each hole. In other alternative embodiments, one hole is formed in the skull, and multiple ultrasound delivery devices are advanced through the hole

The hole (or holes) in the patient's skull may be formed using any suitable devices and methods. For example, in some embodiments a hand or power drill or burr device may be used, such as those commonly known in the art for forming holes in the skull. Once a hole is formed in the skull, one or more ultrasound delivery devices may be advanced partway or completely into the hole or through the hole. In one embodiment, for example, a delivery device is placed into the hole so a distal end of the device is flush with the inner wall of the skull. In other alternative embodiments, one or more delivery devices are advanced through the hole(s) into the epidural space, one or more ventricles and/or an intracerebral space of the patient's brain. For the purposes of this application, “intracerebral space” means any location within brain tissue or parenchyma outside of blood vessels.

To facilitate introduction of ultrasound delivery devices through one or more holes in the patient's skull, one or more introducer devices may optionally be used. For example, in one embodiment an introducer device is placed at least partway into a hole, and at least one ultrasound delivery device is advanced partway or all the way through the introducer device. In one alternative embodiment, the introducer device is advanced through a hole and into the patient's epidural space, and one or more ultrasound devices are thus advanced into the epidural space. In other alternative embodiments, the introducer device may be advanced through the hole and into a ventricle or an intracerebral space of the patient's brain, and one or more ultrasound devices are thus advanced into the ventricle or intracerebral space.

Any suitable ultrasound delivery device may be used in implementing various embodiments of the present invention. For example, in one embodiment, the device may comprise an ultrasound transducer. In another embodiment, the device comprises a transducer-tipped ultrasound catheter. In either case, the ultrasound transducers may be formed from piezoelectric crystal or from silicon-based ultrasonic transducer technology.

In many embodiments, the ultrasound energy is transmitted acutely, such as in treatment of ischemic stroke or acute head trauma. In alternative embodiments, the ultrasound energy may be transmitted chronically, such as in treatment of chronic brain perfusion disorders. In some cases, a device or part of a device may be implanted in the patient for chronic treatment. In various embodiments, any of a number of different conditions may be treated or ameliorated with the methods of the present invention. For example, the ultrasound energy may be transmitted to a blood clot, either within or outside of a blood vessel, to help disrupt the clot. In another embodiment, the energy may be transmitted to a blood vessel to treat atherosclerosis of the vessel. In other embodiments, the energy may be transmitted to one or more blood vessels in the brain to help treat any of a number of blood perfusion abnormalities.

Optionally, the method may further include providing one or more pharmacologic agents to the patient, in conjunction with the delivered ultrasound energy. Examples of such agents include, but are not limited to, tissue plasminogen activator and other blood clot reducing agents, such as tPA, BB-10153, rTPA, Urokinease, Streptase (Streptokinase) Actiase (Alteplase) and Desmoteplase. Other agents which may be used include antiplatelet agents such as aspirin, Plavix (clopidorgel) and Ticlid (Ticclopidine), and GIIb/IIIa inhibitors, such as Reopro (abciximab), Aggrestat (Tirofiban) and Integrilin (eptifibatide). Such a pharmacologic agent may be delivered intravenously, arterially, via intramuscular injection, directly to the blood clot or orally, in various embodiments. Alternative methods optionally involve delivering microbubbles or nanobubbles into the patient's bloodstream, in conjunction with the delivered ultrasound energy. Such microbubbles or nanobubbles may be delivered directly to the blood clot, intravenously or arterially. In some embodiments, both microbubbles or nanobubbles and a pharmacologic agent may be delivered to the patient along with the ultrasound energy. The therapeutic agent and microbubbles may also be delivered to the treatment site through an ultrasound device, an introducer and any suitable catheter.

Once one or more holes have been formed in the skull, ultrasound energy may be transmitted from any of several locations and in any of a number of different patterns. For example, in one embodiment, multiple holes are formed in the patient's skull, and ultrasound energy is transmitted from multiple delivery devices at multiple locations simultaneously. Such a delivery pattern may be advantageous, for example, in triangulating the ultrasound transmissions toward the same target. In an alternative embodiment, ultrasound energy is delivered sequentially from multiple delivery devices. In some cases, the ultrasound energy is transmitted from multiple delivery devices with the same frequency and intensity. Alternatively, the ultrasound energy may be transmitted from multiple delivery devices with different frequencies, different intensities and/or different modes. Ultrasound energy may be transmitted at any desired frequency, although in preferred embodiments the energy has a frequency between about 1 KHz and about 20 MHz, and more preferably between about 17 KHz and about 10 MHz. According to different embodiments, the ultrasound energy may be transmitted in continuous mode or pulse mode or may be modulated.

At any point during or after advancement of an ultrasound device through a hole in the skull, the location of the device may be monitored via any suitable visualization apparatus. For example, radiographic, computed tomography (CT) or magnetic resonance imaging (MRI) technologies may be used to help facilitate placement of an ultrasound delivery device in a desired location. In some embodiments, radiographs, CT images and/or MRI images may be used before device placement to determine an ideal location for the device.

In some embodiments Optical Coherence Tomography (OCT) may be employed. OCT is an optical signal acquisition and processing method, typically employing near-infrared light. The use of relatively long wavelength light allows it to penetrate into the scattering medium. It captures three-dimensional images from within optical scattering media, such as biological tissue or blood clots. Examples of such systems include, but are not limited to devices from Coherent Diagnostic Technology (CDT), LLC, based in Westford, Mass.

In other embodiments, an ultrasound device can work in conjunction with an electronic endoscope system that has a mini camera on the distal end and an additional working channel for therapeutic devices. The working channel of the endoscope may accommodate the ultrasound device and can serve as an outlet to remove blood clots. Examples of such devices include but are not limited to endoscopes from Pentax Medical Company, New Jersey; Olympus America, Center Valley, Pa.; Richard Wollf GmbH, Knittlingen, Germany.

In yet another embodiment, an imaging camera may be provided on the distal end of an ultrasound device. Such electronic imaging camera may have a light emitting source provided by light emitting diodes (LAD) or by light delivered via fiber optics from an external source. The principles of operation are identical to endoscopes, but in this case the camera is incorporated in a single device together with the ultrasound device. Examples of such suitable cameras include but are not limited to devices from MediGus, Ltd, Omer, Israel; OmniVision, Santa Clara, Calif.; Clear Image Technology, Elyria, Ohio.

In another embodiment, an ultrasound device can work in conjunction with a neuro-navigational system. To support the placement of the ultrasound device, an access device may be used. The access device has attributes that enable precise positioning and immobilization of the ultrasound device at a specific angle or range of angles with respect to the skull. The access device can be a part of a stereotaxis frame, or it can be frameless and therefore directly secured to the skull. Examples of such frameless devices include but are not limited to the “Navigus System for Frameless Access” and the NAVIGATION™ products made by Image-Guided Neurologics, Inc., located in Melbourne, Fla., or the Stealth Station™ Intraoperative Guiding system (Medtronic Sofamor Danek, Memphis, Tenn.). Using a stereotaxis frame or a frameless access device, the ultrasound device may be placed on the scalp surface, on the skull surface, inside the skull, or positioned above the skull. For frameless stereotaxy, users can choose between anatomic landmarks (ALs) or surface fiducial markers (FMs) for their match points to define an alignment of the head in the physical and radiographic image space. To further facilitate positioning of the ultrasound device inside the head, fiducial markers may be placed on the ultrasound device or handle assembly that includes ultrasound device.

In some embodiments, during ultrasound energy delivery to the target site in the brain, patient recovery status may be monitored using one or more sensing methods, such as but not limited to monitoring of oxygen levels or saturation, rate of carbon dioxide production, heart rate, intracranial pressure and/or blood pressure. Also, the sensing element's measure could be used to modulate the intensity, frequency and/or duty cycle of the ultrasonic device(s). Such a feedback process is also known as a closed loop control system. Some embodiments may also include the use of a disposable patient interface (DPI), a sterile, compliant conductive gel/oil pack which interfaces between the ultrasound transducer and the patient.

In another aspect of the present invention, a method for delivering ultrasound energy from within a patient's epidural space involves advancing at least one ultrasound delivery device having a visualization feature through at least one hole in the patient's skull to locate at least a distal portion of the device in the patient's epidural space and transmitting ultrasound energy from the ultrasound delivery device(s). Such a method may further involve forming the hole(s) in the patient's skull. According to various embodiments, any of the features or variations of the methods described above may be implemented.

In another aspect of the present invention, a method for delivering ultrasound energy from within at least one ventricle of a patient's brain involves advancing at least one ultrasound delivery device with a visualization guidance through at least one hole in the patient's skull to locate at least a distal portion of the device in at least one ventricle of the patient's brain and transmitting ultrasound energy from the ultrasound delivery device(s). Again, such a method may further include forming the hole(s) in the patient's skull. Any of the features or variations described above may be implemented in various embodiments.

In another aspect of the present invention, a method for delivering ultrasound energy from within an intracerebral space of a patient's brain involves advancing at least one ultrasound delivery device having a visualization feature through at least one hole in the patient's skull to locate at least a distal portion of the device an intracerebral space of the patient's brain and transmitting ultrasound energy from the ultrasound delivery device(s). The method may further include forming the hole(s) in the patient's skull. And again, any of the features or variations described above may be implemented, according to various embodiments. Delivery of ultrasound energy from the intracerebral space may be used for treatment of any of a number of conditions, such as acute clot outside of blood vessels caused by brain trauma or ischemic stroke caused by a clot within a vessel. In various embodiments, ultrasound may be combined with delivery of a pharmacological agent, microbubbles/nanobubbles or both. Ultrasound, with or without additional agents, may be delivered until the patient's symptoms improve and/or until a brain imaging study (e.g. MR, CT, PET, SPECT) demonstrate that the adverse “mass effects” of a clot outside are significantly reduced (e.g., <10% in size) and removed. Dissolved blood clots maybe removed outside the head using one of the following methods: (i) aspiration methods with a simple syringe or other similar devices; (ii) drainage by gravity by positioning a collection bag bellow the head: or (iii) a combination of both approaches. Dissolved blood clots maybe removed outside the head via an ultrasound device, an introducer, or any other suitable catheter.

In yet another aspect of the present invention, a method for dissolving and recanalizing blockages in intracranial drainage catheters, cannulas or introducers using ultrasound energy is provided. Ultrasound energy delivery may be used to recanalize or dissolve blockages created by cerebrospinal fluid or blood clots. According to various embodiments, cerebrospinal fluid or blood clots may be located inside or outside of the drainage catheter, cannula or introducer. Also, one or more pharmacologic agents or microbubbles or nanobubbles may be delivered to the catheter to further facilitate the dissolving and recanalization process.

A method for removing blockages in a drainage catheter or introducer that is placed in the treatment area of a patient's intracranial space using ultrasound energy involves forming at least one aperture in the patient's skull; positioning the drainage catheter cannula or introducer through the aperture to a desired location within the intracranial space; advancing an ultrasound device having a distal portion into the drainage catheter, cannula or introducer, and transmitting ultrasound energy at frequencies between 1 KHz and 20 MHz from the ultrasound device. Such a method may further include advancing the ultrasound device into the drainage catheter, cannula or introducer through the proximal end of the drainage catheter, or through a side hole in the drainage catheter.

In one embodiment, the ultrasound device can be moved back and forth within the drainage catheter, cannula or introducer and outside the drainage catheter, cannula or introducer, to dissolve cerebrospinal fluid blockages or blood clots located either inside or outside of the drainage catheter, cannula or introducer.

In another embodiment, the ultrasound device may be permanently implanted inside the drainage catheter and connected to the energy source on an as-needed basis when required to remove blockages.

Another embodiment includes removing blockages of cerebrospinal fluid, blood clots or combination of both, to outside of the drainage catheter, cannula or introducer using aspiration, drainage by gravity, or combination of both.

In yet another embodiment, the ultrasound device may be positioned in multiple drainage catheters, cannulas or introducers that are further positioned in multiple treatment locations.

For treatment of clots inside a vessel, as in ischemic stroke patients, the ultrasound delivery device may be placed near or directly adjacent to the clotted blood vessel. For treatment of clots outside the vessel, the ultrasound delivery device may be placed near or inside the clot.

Another embodiment includes removing blood clots or other tissue from the patient's head that is located outside the burr hole or twist hole access. In such cases, blood clots or tissue to be removed may be located outside a small burr/twist hole in the head, and devices with the ability to deflect inside the head beyond the burr hole longitudinal aperture plane may be utilized.

Further aspects and embodiments of the present invention are described in greater detail below, with reference to the attached drawing figures.

### BRIEF DESCRIPTION OF THE DRAWINGS

FIG. 1 is a cross-sectional view of a portion of a human skull, showing the skull, brain tissue and epidural space and a hole formed in the skull with an introducer device in place, according to one embodiment of the present invention.

FIG. 2A is a cross-sectional view as in FIG. 1, with multiple ultrasound delivery devices advanced through the introducer device into the epidural space, according to one embodiment of the present invention.

FIG. 2B is a view of the introducer device and ultrasound delivery devices of FIG. 2A.

FIG. 2C is a cross-sectional view as in FIG. 1, with multiple catheter-based ultrasound delivery devices advanced through the introducer device into the epidural space, according to an alternative embodiment of the present invention.

FIG. 3 is a cross-sectional view of a human skull with a hole formed therein and with an ultrasound transducer device in place within the hole, according to an alternative embodiment of the present invention.

FIG. 4 is a cross-sectional view of a human skull and brain, showing an ultrasound delivery device advanced through a hole in the skull and into a ventricle of the brain, according to one embodiment of the present invention.

FIG. 5 is a cross-sectional view of a human skull and brain, showing an ultrasound delivery device advanced through a hole in the skull and into a ventricle of the brain, according to an alternative embodiment of the present invention.

FIG. 6 is a frontal diagrammatic view of a human torso and head, demonstrating an implantable ultrasound delivery system for chronic treatments, according to one embodiment of the present invention.

FIG. 7 is a side view of a human head with three ultrasound transducers coupled therewith, demonstrating a triangulation technique for delivering ultrasound energy to a location in the brain, according to one embodiment of the present invention.

FIG. 8 is a cross-sectional view of a human skull and brain, showing an ultrasound delivery device advanced through a hole in the skull and into an intracerebral space of the brain, according to an alternative embodiment of the present invention.

FIG. 9 is a cross-sectional view of a human skull and brain, showing a drainage catheter advanced through an introducer in a hole in the skull and into an intracerebral space of the brain where blood clots are located. An ultrasound delivery device is advanced through an introducer and into a side opening inside the drainage catheter distal part.

FIG. 10 is a cross sectional view of a human skull and brain, showing an ultrasound device advanced through an introducer in a hole in the skull and into an aneurysm filled with a blood clot.

FIG. 11 illustrates an apparatus for removal of blood clots or other tissue from the patient's head that includes visualization, therapeutic and aspiration features.

FIG. 12a illustrates the handle assembly of the apparatus shown in FIG. 11 having an internal pre-shaped probe in a straight configuration.

FIG. 12b illustrates the handle assembly of FIG. 12a having the internal pre-shaped probe in a deflected position.

FIG. 13a is a cross sectional view of the handle assembly of FIG. 12a advanced through the skull to location where blood clots are located.

FIG. 13b is a cross sectional view of the handle assembly of FIG. 12b located inside the skull where blood clots are located.

### DETAILED DESCRIPTION OF THE INVENTION

Methods and apparatus of the present invention generally involve delivering ultrasound energy to a patient's intracranial space for diagnostic purposes, or therapeutic treatment, or both. The methods involve forming at least one hole in the patient's skull, advancing at least one ultrasound delivery device at least partway through the hole(s), and transmitting ultrasound energy from the ultrasound delivery device(s). In some instances, such as in treatment of ischemic stroke, ultrasound energy is delivered to a target clot in a blood vessel. In other cases, such as in acute head trauma, ultrasound energy may be directed toward an extravascular blood clot in the brain (often referred as intracranial hemorrhage or ICH). In other cases, energy may be delivered toward an area of blood vessels to cause vasodilatation and thus increased blood flow. Thus, the techniques and apparatus described herein may be used for a number of different applications and treatments and are not limited, for example, to treatment of an isolated intracranial blood clot or even to ischemic stroke therapy.

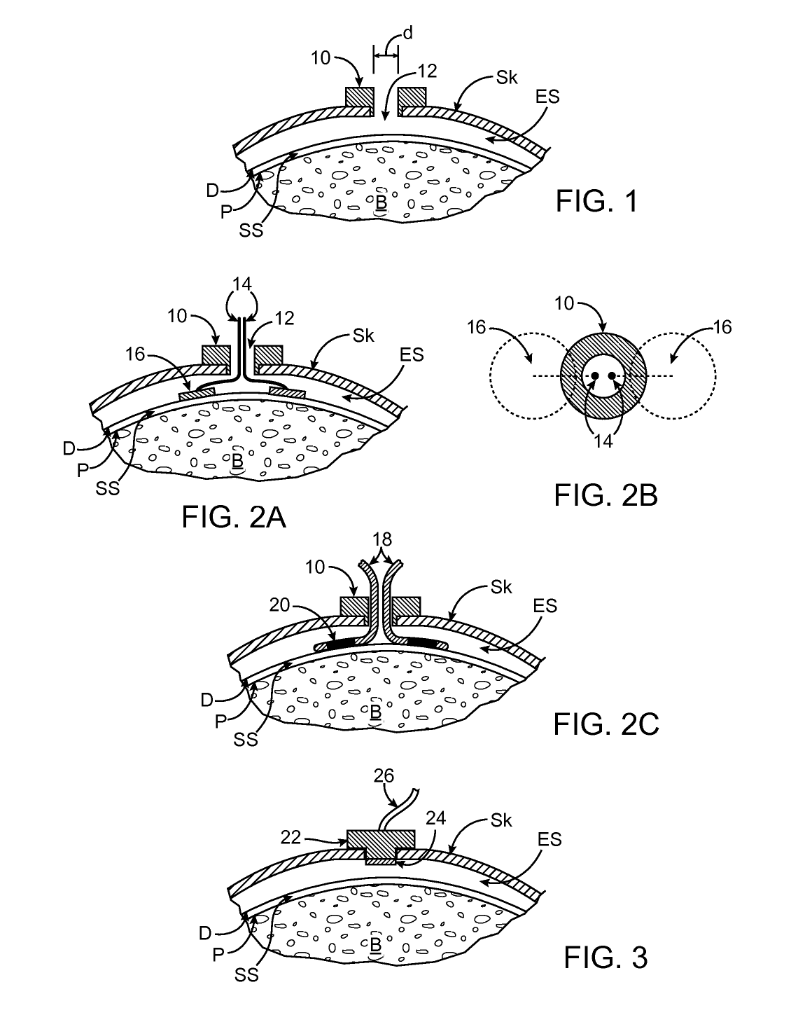

With reference now to FIG. 1, a cross-sectional view of a portion of a human head is shown, with a skull Sk, epidural space ES, dura mater D, subarachnoid space SS, pia mater P and brain tissue B. In various embodiments, one or more holes 12 or openings are formed in the skull Sk using any suitable hole forming device, such as but not limited to a power drill, hand drill, or burr device. In some embodiments, a guide device 10 (or “introducer”) is placed in hole 12 to facilitate delivery of one or more ultrasound delivery devices. In alternative embodiments, guide device 10 is not used. Hole(s) and the opening of guide device 10 may have any desired diameters. For example, the opening of guide device 10 may have a diameter d ranging from about 0.5 mm to about 20.0 mm in one embodiment.

Guide device 10 may be attached to the skull Sk by any suitable means. In some embodiments, for example, guide device 10 is pressure fitted within hole 12, while in other embodiments guide device 10 may have threads for screwing into hole 12 or may include a locking mechanism for attaching to the skull Sk. In some embodiments, one or More atraumatic guide catheters (not shown) may be used with guide device 10 to introduce one or more ultrasound delivery devices into hole 12 or into the epidural space ES. Use of such a guide catheter may help ensure that no intracranial structures are damaged.

Referring now to FIGS. 2A and 2B, in one embodiment, two ultrasound delivery leads 14, each having a transducer 16 (or “ultrasound wand”) coupled to its distal end, may be delivered through guide device 10 into the epidural space ES. Transducers 16 may then rest on the dura mater D or float within the epidural space ES, and ultrasound energy may then be transmitted from the wands into the intracranial space. Transducers 16 may be delivered through a microcatheter or via any other suitable delivery technique. Furthermore, any number of ultrasound delivery leads 14 and transducers 16 may be delivered through hole 12, such as from one to ten leads 14 and transducers 16. FIG. 2B shows introducer 10, leads 14 and transducers 16 from a top view.

Referring now to FIG. 2C, an alternative embodiment is shown in which multiple ultrasound catheters 18 are delivered through hole 12 into the epidural space.ES. Each ultrasound catheter 18 includes a distal ultrasound transducer 20, which transmits ultrasound energy into the intracranial space. Again, any number of catheters 18 may be introduced through one hole, such as anywhere from one to ten catheters 18. Catheter 18 may be an over-the-wire or not over-the-wire, in various embodiments. Each catheter 18 may include one ultrasound transducer 20 or may include multiple transducers 20 distributed along its distal portion. In one embodiment, a distal portion of catheter 18 may have a straight configuration when being delivered but may then assume a helical shape when deployed in the epidural space ES, with the helix having a larger diameter than hole 12. The helical portion may then contain multiple transducers to allow transmission of ultrasound in multiple different directions. Catheters 18 also have a deflectable tip to allow it to be moved to various locations within the epidural space ES without causing damage. Transducers 20 may be formed from piezoelectric crystal or using chip technology. In some embodiments, for example, transducers 20 may be fabricated on the surface of a silicon wafer.

With reference now to FIG. 3, in an alternative embodiment, no introducer or guide device is used. Instead, an ultrasound transducer 22 with an ultrasound delivery tip 24 and coupled to a power supply via a lead 26 is placed directly within hole 12 in the skull. Transducer 22 may extend only partway into hole 12 or alternatively may extend all the way into hole 12 or even extend into the epidural space ES, as shown. Transducer 22 is then used to deliver ultrasound energy to the intracranial space.

In any of the embodiments described above, any desired number of holes 12 may be formed in the skull Sk and any desired number of ultrasound delivery devices may be inserted into the holes to deliver ultrasound energy. For example, in some embodiments one hole 12 is formed and one delivery device is used. In another embodiment, one hole 12 may be formed and multiple delivery devices inserted through that hole 12. In other alternative embodiments, multiple holes 12 are formed and either one or multiple delivery devices may be placed through each hole. As described further below, forming multiple holes and using multiple ultrasound delivery devices may be advantageous in some cases in that it allows for the delivery of ultrasound energy from multiple angles simultaneously or in succession.

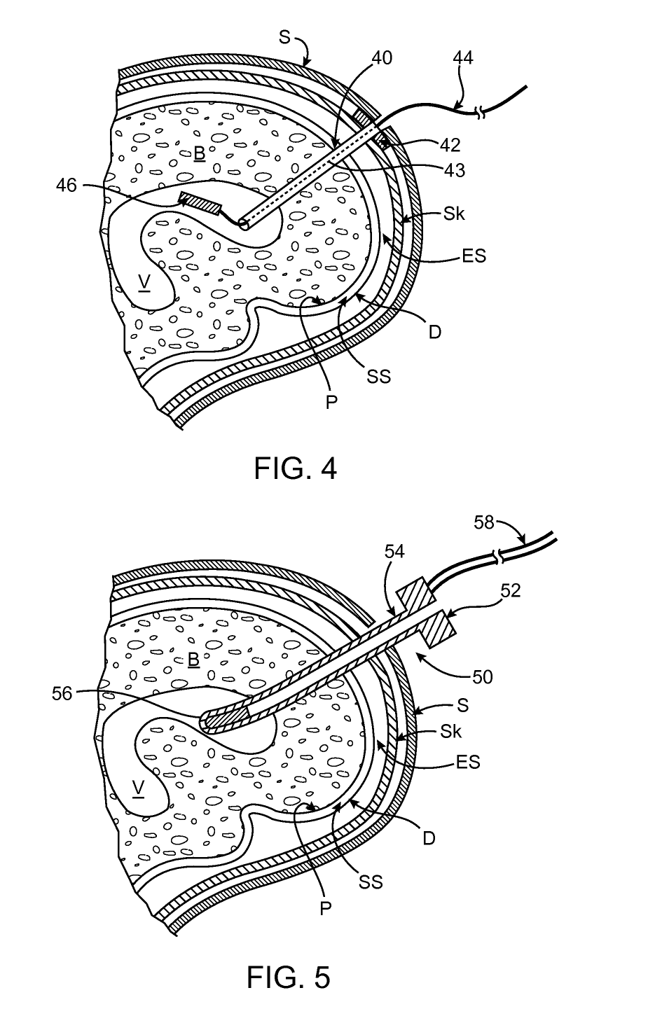

Referring to FIG. 4, in some embodiments, a catheter device 40 may be used to advance an ultrasound delivery wand 46 into a ventricle V of a brain B. In one embodiment, catheter 40 includes a hub 42, a catheter shaft 43, a lead 44 and wand 46 attached to the distal end of lead 44. As shown, catheter 40 extends through the scalp S, skull Sk, epidural space ES, dura mater D, subarachnoid space SS, pia mater P and brain tissue B to enter the ventricle V. Hub 42 may rest under the scalp S, as shown, or on top of the scalp S, in various embodiments. Catheter 40 is fully retrievable, so that the wand 46, lead 44, catheter shaft 43 and hub 42 may be easily removed from the patient. Delivering ultrasound energy from within a ventricle V in the brain B may be very advantageous in some cases, depending on the location of the target treatment area.

With reference now to FIG. 5, an alternative embodiment of an ultrasound delivery device 50 for delivering energy from within a ventricle V is shown. In this embodiment, delivery device 50 includes a hub 52, a catheter shaft 54, a wand 56 at or near the distal end of catheter shaft 54, and a lead 58 coupling device 50 to a power supply. In some embodiments, catheter shaft 54 is steerable, to facilitate delivery of wand 56 into the ventricle V. In various embodiments, hub 52 may reside either outside or inside the scalp S.

In either of the intraventricular approaches just described, or in any other intraventricular approach, the catheter may be placed blindly, via bony landmarks, into one of the ventricles of the brain via a traditional ventriculostomy approach. After forming a hole in the skull, the catheter or guidewire system is placed into the ventricle. When clear cerebrospinal fluid flows out of the proximal end of the catheter, the physician knows the distal end of the catheter is in the ventricle. In an alternative embodiment, intraoperative computed tomography (CT) imaging may be used to help guide placement of the catheter. In another embodiment, preoperative CT and/or MRI scanning may be used with an image-guided system to help guide the catheter into the ventricle. Such image guided systems are provided, for example, by Medtronic, Inc. (StealthStation S7), or BrainLAB, Inc. (VectorVision System). Once the catheter is placed in the ventricle, one or more transducers may be advanced through the catheter, as in the embodiment shown in FIG. 4. Alternatively, one or more transducers may be included at or near the distal end of the catheter, as in the embodiment shown in FIG. 5.

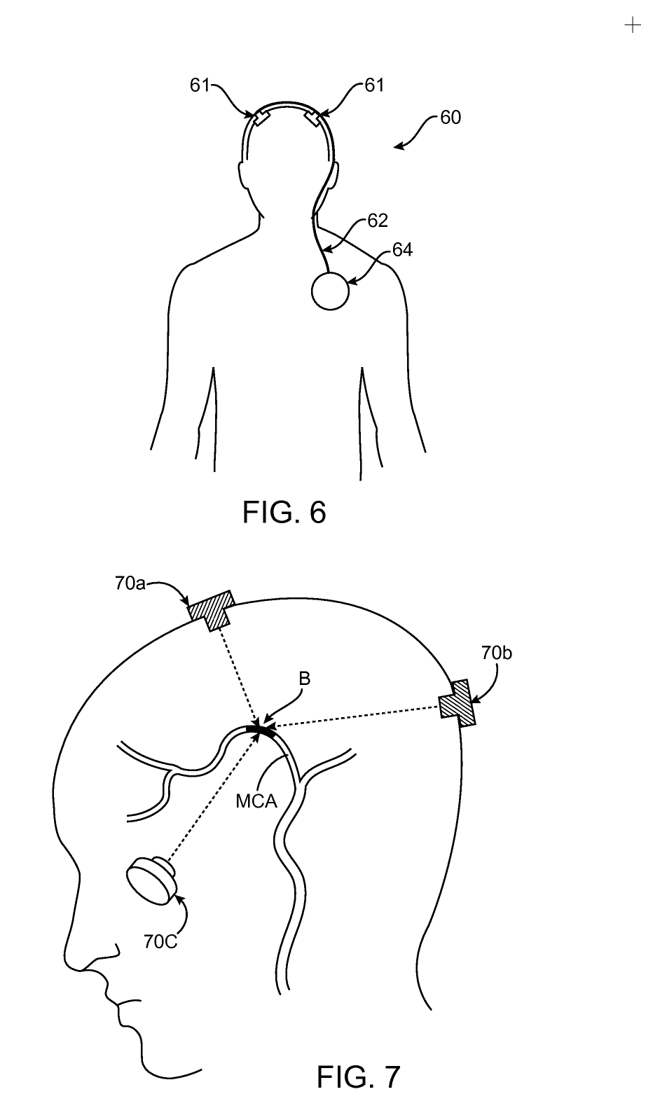

Referring now to FIG. 6, for some treatments it may be desirable to implant one or more ultrasound delivery devices in a patient and use the devices for chronic therapy. Such implantable devices may be used, for example, in treating Alzheimer's disease or a chronic brain perfusion disorder, or in increasing perfusion over time to enhance brain function. In one embodiment, an implantable ultrasound delivery system 60 includes multiple ultrasound delivery devices 61, coupled with multiple leads 62, which may be tunneled under the scalp and skin to an implanted power source 64 in the chest. In an alternative embodiment, power source 64 may be implanted under the patient's scalp or even inside the patient's skull. One type of intracranial implantable power supply, for example, is provided by Neuro Pace, Inc. Types of implantable power sources include standard lithium ion non-rechargeable or rechargeable batteries. In an another alternative embodiment, the power source 64 could be located external to the body and would transmit the power to an implanted receiver coil in the patient via radio frequency energy. The implantable receiver coil would convert the power into the appropriate form and be connected to the ultrasound system wires. Ultrasound delivery devices 61 may then deliver continuous or intermittent ultrasound energy to one or more intracranial target areas to enhance blood flow. Each device 61 is placed within a hole formed in the skull.

As mentioned above, and with reference now to FIG. 7, in some embodiment multiple ultrasound delivery devices 70 are placed in multiple holes in a patient's skull to deliver ultrasound energy to an intracranial target area from multiple angles. In the embodiment shown, three delivery devices 70a-70c are used to direct energy toward a blockage B in the middle cerebral artery MCA. Triangulation of ultrasound energy signals in this way typically enhances the ability of the energy to break up a blockage B. In embodiments where multiple ultrasound devices 70 are used, energy may be transmitted from devices 70 either simultaneously or at different times. In some embodiments, for example, energy may be transmitted sequentially.

In one embodiment of the triangulation method described by FIG. 7, preoperative CT/CTA (computed tomography angiography) and/or MR/MRA (magnetic resonance angiography) images are obtained of the patient's intracranial space. These images are obtained with some type of fiduciaries on the patient's head, such as screw-on or stick-on fiduciaries. Once the images are obtained and a clot location identified, computer software may be used to recommend where to locate the ultrasound delivery devices on the skull or within the epidural space or ventricular space(s). Based on the software recommendations, multiple delivery devices are then placed, typically though not necessarily three or more devices. The ultrasound transducer(s) could be made of MR and/or CT compatible materials so that the related heating or imaging artifacts are minimized during scans. It is important that transducer(s) is made of MR/CT compatible materials because patients with acute stroke may need to be imaged, scanned multiple times to access recovery progress.

In any of the embodiments described above, any desired ultrasound frequency and intensity may be delivered, and ultrasound energy may be delivered in continuous mode, pulsed mode, or a combination thereof. In various embodiments, for example, ultrasound frequencies of between about 1 KHz and about 10 MHz may be used. When pulse mode is used, the pulse mode may vary from about 1% to about 99% of the duty cycle.

Additionally, in various embodiments, ultrasound energy may be delivered along with intravenous or intraarterial drug delivery and/or intravenous delivery of microbubbles or nanobubbles. For example, ultrasound may be delivered along with tissue plasminogen activator and other blood clot reducing agents, such as tPA, BB-10153, rTPA, Urokinease, Streptase (Streptokinase) Actiase (Alteplase) and Desmoteplase. Other agents which may be used include antiplatelet agents such as aspirin, Plavix (clopidorgel) and Ticlid (Ticclopidine), and GIIb/IIIa inhibitors, such as Reopro (abciximab), Aggrestat (Tirofiban) and Integrilin (eptifibatide). Microbubbles or nanobubbles of lipids or other suitable substances may also be used.

Once a procedure is completed and the ultrasound delivery device(s) are removed, the hole(s) in the skull may be filled using any suitable technique, such as with known techniques using plugs or bands.

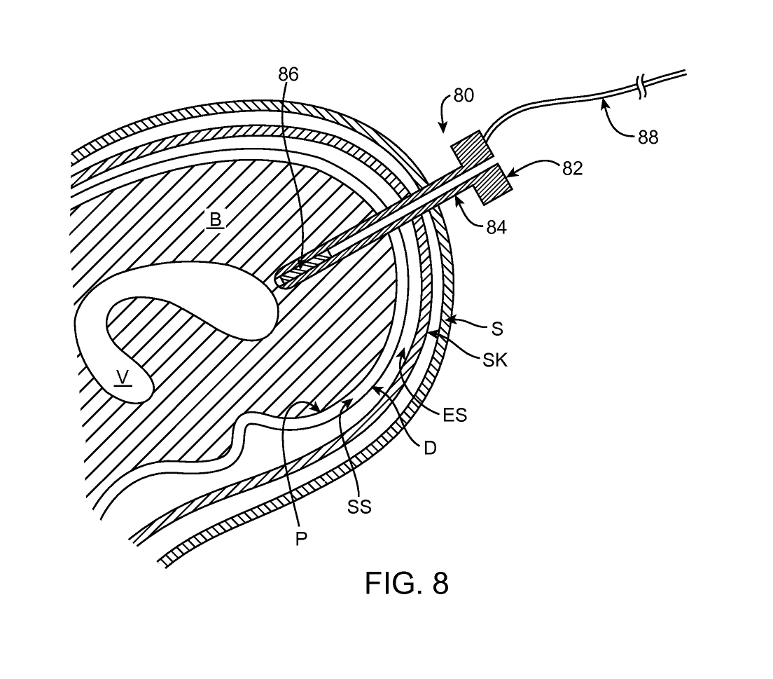

Referring now to FIG. 8, in some embodiments, a catheter device 80 may be advanced through a hole in a patient's skull Sk so that an ultrasound transducer 86 of device 80 is located in the intracerbral space of the patient's brain B. In one embodiment, catheter device 80 includes a hub 82, a catheter shaft 84, ultrasound transducer 86, and a lead 88 connecting device 80 to a power supply. As shown, catheter device 80 extends through the scalp S, skull Sk, epidural space ES, dura mater D, subarachnoid space SS, and pia mater P and into brain tissue B. Hub 82 may reside outside the scalp S, as shown, or under the scalp S, in various embodiments. Catheter device 80 is fully retrievable. Delivering ultrasound energy from within the intracerebral space in the brain B may be very advantageous in some cases, such as in treatment of acute hemorrhage and/or clot caused by head trauma.

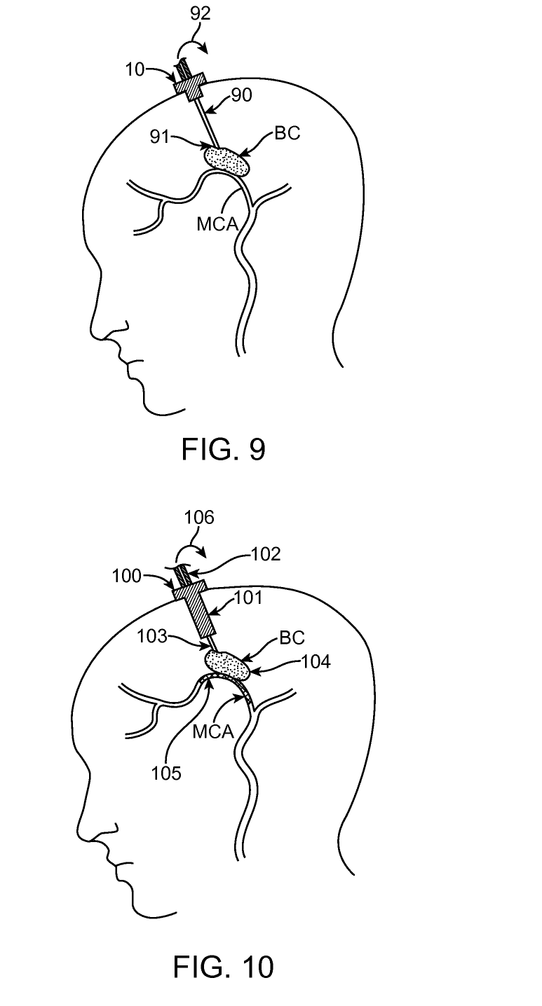

Referring now to FIG. 9, an alternative embodiment is shown in which a drainage catheter 90 is positioned through the introducer 10 in the patient's intracerebral space. The distal portion 91 of the drainage catheter 90 (which can have a series of side holes (not shown)) is placed inside blood clots (BC).

An ultrasound device 92 is placed inside the drainage catheter 90, with the distal end of the ultrasound catheter 92 positioned within the distal end 91 of the drainage catheter 90. The distal end of the ultrasound catheter 92 may be positioned at any location within the drainage catheter 90, or it may be moved within the drainage catheter 90 during ultrasound energy delivery. Examples of drainage catheters include but are not limited to catheters manufactured by Codman, Johnson & Johnson Company located in Raynham, Mass. Examples of introducers include but are not limited to such devices also manufactured by Codman.

Alternatively, the ultrasound catheter 92 may be placed through the introducer 10 in parallel to the drainage catheter 90, and its distal end maybe placed through side hole(s) onto the distal end of the drainage catheter 91 (not shown). Activation of the ultrasound catheter 92 will cause ultrasound energy propagation inside the drainage catheter that will consequently dissolve cerebrospinal fluid blockages or blood clots located either inside or outside of the drainage catheter 90. To facilitate removal of dissolved cerebrospinal fluid blockages or blood clots from the drainage catheter 90 outside of the head, aspiration or gravitational methods may be employed. Additionally, if the ultrasound catheter 92 requires irrigation while delivering ultrasound energy, irrigation medium and cerebrospinal fluid blockages or blood clots may be removed outside the head using the same irrigation methods.

The drainage catheter 90 may be repositioned within the blood clots BC, or it may be repositioned into blood clots located in different areas of the brain, including the intracerebral location of the intracranial space, the ventricle of the patient's brain, or underneath the dura mater located within the intracranial space or in the epidural location of the intracranial space.

To further improve the ability to dissolve cerebrospinal fluid blockages and blood clots, delivery of one or more pharmacologic agents to the drainage catheter 90, or microbubbles or nanobubbles, may be helpful. Such pharmacologic agents, microbubbles or nanobubbles can be delivered directly or in mixture with a conventional saline to the treatment location

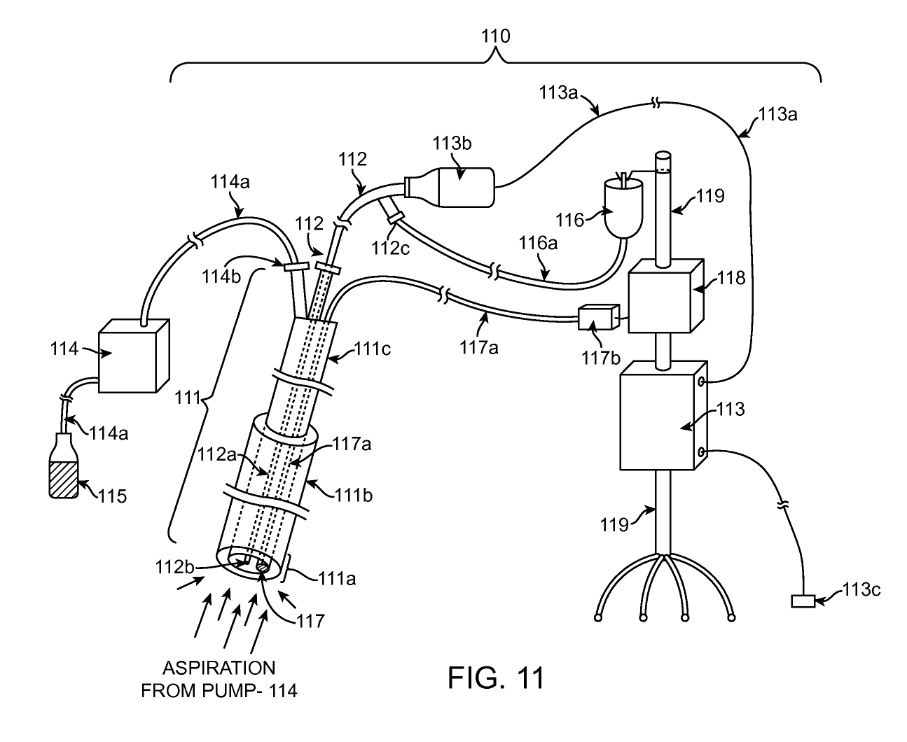

FIG. 10 is a cross sectional view of a human skull and brain, showing an ultrasound device 102 having the distal portion 103 placed through an introducer 100, with the introducer 100 having a distal portion 101 positioned in a hole in the skull and into an aneurysm 104 filled with blood clot BC. The distal end of the ultrasound catheter 103 may be forced through the wall of the aneurysm 104 into the blood clot BC. Alternatively, the aneurysm wall may be cut or opened with knives, scissors, scalpels or any other suitable surgical tool, and then the distal portion 103 of the ultrasound device 102 is placed inside. The introducer 100 may be advanced to the treatment location prior to advancing the ultrasound device 102. Also, if desired, the introducer 100 may be removed from the hole in the skull after placing the ultrasound device 102 within the aneurysm 104.

A blood diverting device 105 shown inside the Middle Cerebral Artery (MCA) is occluding blood access to the aneurysm 104, so there is no addition blood supply reaching inside the aneurysm 104. The diverting device 105 may be in the form of a self-expandable stent with a tight strut configuration, or a covered stent that is either self expandable or balloon expandable, so that blood flow along the MCA is well maintained while preventing more blood penetration into the aneurysm 104. The diverting device 105 can be introduced into the MCA using known techniques, such as less invasive endovascular method, similar to one used in delivery of intracranial coils or stents. Examples of such diverting devices 105 include but are not limited to the Pipeline Embolization Device manufactured by EV3 Endovascular (Plymouth, Minn.).

Activation of the ultrasound device 102 will deliver ultrasound energy to the aneurysm location and dissolve the blood clots BC located inside the aneurysm 104. To facilitate removal of dissolved blood clots BC to the outside of the skull, known aspiration or vacuum methods may be employed as shown with arrow 106. Additionally, if the ultrasound catheter 102 requires irrigation while delivering ultrasound energy, irrigation medium and blood clots may be removed outside the skull using the same irrigation methods. The ultrasound device 102 may be repositioned within the aneurysm 104 to assure removal of the majority of blood clots BC.

FIG. 11 is an overview of an apparatus 110 for removal of blood clots or other tissue from the patient's head. The apparatus 110 includes the following major components: a handle assembly 111 having a distal end 111a, an external shaft 111b and an internal shaft 111c; an ultrasound catheter 112 having an energy transmission wire 112a, a distal end 112b and an irrigation port 112c; an ultrasound generator 113 connected via a cable 113a with a transducer 113b, and a system activation foot-switch 113c; a vacuum/aspiration pump 114 with an aspiration tube 114a connected to an aspiration port 114b and a container 115 for collection of aspirated blood clots/tissue; a saline bag 116 with a catheter irrigation tube 116a; a mini camera 117 with a camera cable 117a that is connected to a circuitry box 117b; and a monitor 118. The circuitry box 117b includes circuitry for signal processing, and may be integrated together with the monitor 118. An IV pole 119 may be used for convenience to put all or some components of the apparatus 110 together. The mini camera 117 and ultrasound catheter 112 described above may be provided together in a single handle assembly 111, or may be provided separately. Accordingly, both the mini camera 117 and the ultrasound catheter 112 may be introduced to the patient's head together, or separately.

The distal portion 111a of the handle assembly 111 is placed through a burr hole or other aperture in the skull and into the area where blood clots or tissue to be removed is located. Positioning of the distal end 111a of the handle assembly 111 inside the head and into the treatment area is usually done based on the previous CT images and/or MRI images, so a neurosurgeon performing this procedure knows the location of the treatment area. For better imaging accuracy, the handle assembly 111 is equipped with a forward-looking mini camera 117 located at the very distal end 111a of the handle assembly 111. Such a camera can help to differentiate blood clots or tissue to be removed from the healthy brain tissue inside the head. The mini camera 117 visualization inside the head is crucial to identify and ensure the presence of the distal end 111a of the handle assembly 111 within the treatment area to further recognize tissue or blood clots to be removed and avoid removal of healthy tissue from the brain. The mini camera 117 is connected via a cable 117a to the circuitry box 117b and monitor 118 that allows the surgeon to view the surrounding area around the distal end 111a of the handle assembly 111 during the removal of blood clots or other tissue. Once blood clots or tissue to be removed are identified by the mini camera 117 on the monitor 118, the apparatus 110 is turned “ON”, which will activate the aspiration pump 114 and the ultrasound catheter 112. The ultrasound catheter 112 is activated by the ultrasound transducer 113b that converts electrical energy from the generator 113 into high frequency mechanical vibrations that are propagated in ultrasonic waveform through the catheter 112 to its distal end 112b. The ultrasound catheter 112 delivers vibrational energy through the transmission wire 112a to its distal end 112b located at the distal end 111a of the handle assembly 111. Blood clots or tissue to be removed are extracted by activation of the aspiration pump 114 and dissolved by the vibrating end 112b of the ultrasound catheter 112. The distal end 112b of the ultrasound catheter 112, while vibrating; liquefies blood clots or other tissue which is being aspirated by the vacuum pump 114 and transported via the tube 114a to the container 115. The vacuums pump 114 may be an independent unit or may include a peristaltic pump device integrated with the ultrasound generator (not shown). During activation of the apparatus 110, conventional saline irrigation from the bag 116 through the catheter 112 may be provided to reduce heat created by ultrasound energy transmission and to further facilitate the aspiration process.

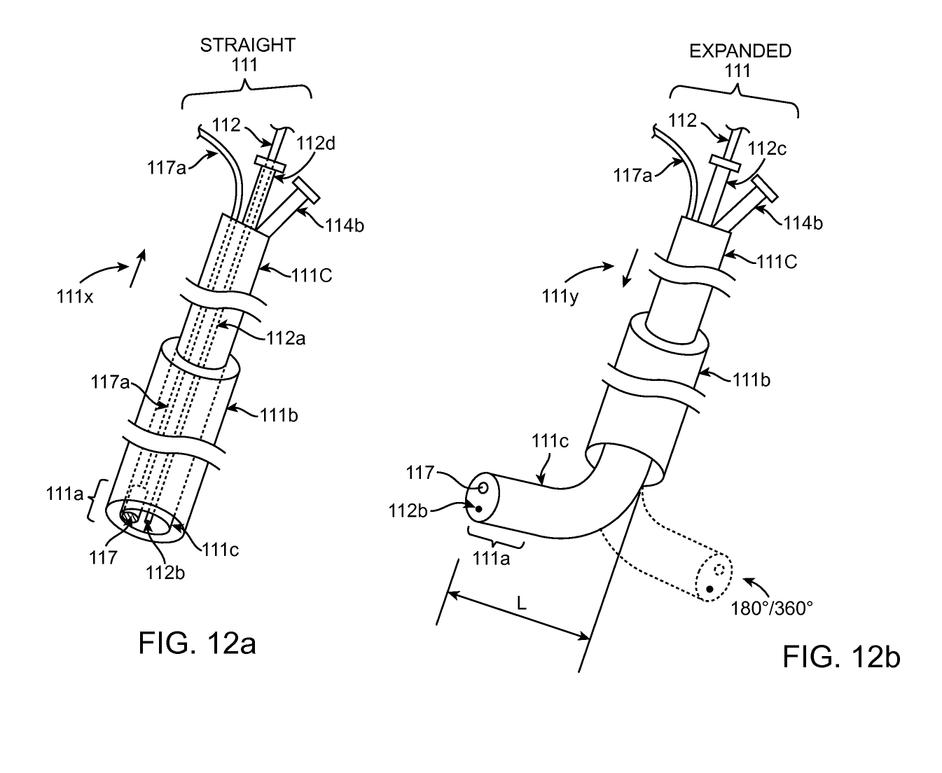

FIG. 12a is an enlarged view of the handle assembly 111 shown in FIG. 11 in a straight position. The handle assembly 111 has an internal shaft 111c which is located inside the external shaft 111b. While the external shaft 111b is straight and shorter, the internal shaft 111c is pre-shaped and longer, and has several ports at its proximal end, including ultrasound catheter port 112d, aspiration port 114b and camera cable 117a outlet. The arrow 111x indicates that the distal end 111a of the internal shaft 111c is inside the external shaft 111b. When the internal shaft 111c is inside the external shaft 111b, it is straightened by the external shaft 111b. However, when the internal shaft 111c is moved outside the external shaft 111b, it will revert to its pre-shaped configuration, reaching areas outside of the external shaft 111b as shown on FIG. 12b. The arrow 111y in FIG. 112b indicates that the distal end 111a of the internal shaft 111c is moved outside of the external shaft 111b.

Both shafts 111b, 111c can be made of metal, polymer or a combination of both, in a variety of configurations including but not limited to tubular, oval, rectangular, or combinations of all. A pre-shaped feature of the distal end of the internal shaft 111c can be achieved using a shape memory alloy such as nickel titanium (NiTI) or shape memory polymers (SMPs), also called “smart” materials that can switch between two shapes, from a fixed (temporary) shape to a predetermined permanent shape. Also, any combination of metal, shape memory metal, shape memory polymers or conventional polymers may be utilized to achieve the desirable deflection characteristics. Shape memory polymers may include, but are not limited to, polyurethanes or polystyrenes. Such memory material(s) are beneficial when there is a need for a device that needs to deflect to access desirable treatment areas that are located in spaces beyond the external shaft 111b.

The very distal part of the handle assembly 111a includes a mini camera 117 and the distal end 112b of the wire 112a. The distal portion of the ultrasound catheter 112 may alternatively include a bare ultrasound transmission wire 112a located on the proximal end of the catheter 112. Other examples of suitable ultrasound catheter configurations for such an application are shown in U.S. Pat. Nos. 4,870,952 (Don Michael et al.); 4,920,954 (Alliger et al.); 5,267,954 (Nita); 5,397,301 (Pfluger et al); 7,137,963 (Nita et al.)

FIG. 12b is an enlarged view of the handle assembly 111 shown in FIG. 11 having an internal shaft 111c in a deflected position. The deflection may be lateral or sideways, outside of the external shaft 111b. The distal end 111a of the internal shaft 111c now extends laterally away from the external shaft 111b. The degree of deflection or distance L as shown in FIG. 12b can be moved and adjusted as desired by moving the internal shaft 111c further in the direction indicated by arrow 111y. The entire handle assembly 111 can be rotated by 360 degrees, so that rotating the distal end 111a will cause the distal portion 111a to access an area within much larger space. The handle assembly 111 can be moved up and down within the patient's head, thereby deflecting the distal end 111a to a wide range of space well beyond its original position as shown in FIG. 12a. Such a deflection feature of the internal shaft 111c can be achieved by using the methods described above. The deflection of the distal end 111a may be also accomplished by utilizing the superelastic property of the ultrasound transmission wire 112b. Such transmission wires are often made of nickel titanium alloys that exhibit shape memory properties. By pre-shaping the distal portion of the ultrasound transmission wire 112a at a preferable angle (not shown), deflection of the internal shaft 111c (which can be made of a polymer material or thin hypotube) may be also attained in desirable configurations when the internal shaft 111c is moved outside of the external shaft 111b as indicated by arrow 111y.

The ability to re-position the distal 111a end of the handle assembly 111 within the patient's head may be achieved in any other suitable manner, including but not limited to using deflection, actuation or angulations of a pre-shaped telescopic member, or combination thereof.

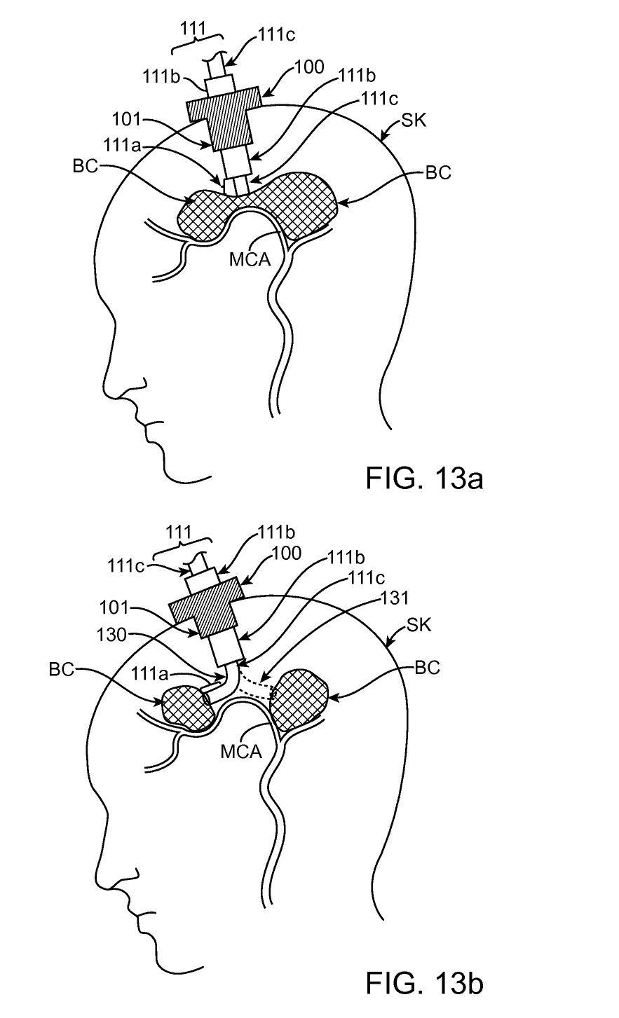

FIG. 13a illustrates the handle assembly 111 in a straight configuration as shown in FIG. 12a placed inside the skull SK to an area where blood clots BC are located. The handle assembly 111 is placed through the introducer 100 having a distal portion 101. The introducer 100 may be placed through a guide device attached or fixed to the skull SK (not shown) that is placed into a burr hole in the skull SK. Upon activating the mini camera 117 and its visualization guidance feature, the distal end 111a of the handle assembly 111 is positioned within the blood clots BC. Once the blood clots BC are visible on the screen 118 (FIG. 11), the ultrasound and aspiration features of the apparatus 110 are activated, and the process of removing blood clots BC begins. As shown in FIG. 13a, the handle assembly 111 is in a straight, non-deflected position having direct access to blood clots within the longitudinal plane along the burr hole. The use of the introducer 100 serves to access an area of the brain where clot removal is required. Use of an introducer together with a dilator is recommended since these devices are much less traumatic in accessing a desirable area of the brain compared with therapeutic devices, in this case, the handle assembly 111. Often, such introducers are peel-away devices and may be removed during the therapy if necessary. Examples of such introducers include but are not limited to Peel-Away Catheter Introducer by Codman, Johnson and Johnson Company (Raynham, Mass.). By moving activated handle assembly 111 longitudinally up and down, the neurosurgeon can remove blood clots within a very limited area as shown in FIG. 13a.

FIG. 13b is a cross sectional view of the internal shaft 111c of the handle assembly 111 that is deflected as shown in FIG. 12b and located inside the skull where blood clots BC are located. The handle assembly 111 and its distal end 111a can be rotated 360 degrees and moved up and down within the burr hole (and introducer 100). With such motion and deflected distal segment of the internal shaft 111c, the distal end 111a of the handle assembly 111 can access areas in the brain well beyond the longitudinal plane of the burr hole. The position 130 of the distal end 111a can access the far left side of the brain where the other part of blood clot BC is located. The position 131 of the distal end 111a can access the far right side of the brain where yet another part of blood clot BC is located. As a result, the deflectable handle assembly 111 can cover the entire space where blood clots BC are located.

To further improve the ability to dissolve blood clots BC, delivery of one or more pharmacologic agents or microbubbles or nanobubbles to the clot location may be helpful. Such pharmacologic agents, microbubbles or nanobubbles can be delivered directly or in mixture with a conventional saline to the treatment location.

Cerebral temperature has been recognized as a strong factor in ischemic brain damage. Clinical evidence has shown that hypothermia ameliorates brain damage. Also, a therapeutic cooling to 30° C.-35° C. that includes the patient head or a whole body (systemic cooling) may reduce ischemic brain damage; reduce intracranial pressure and edema after ICH. Focused cranial cooling can be achieved with a simple method of placing ice or cold gel packs around the head or neck. Systemic cooling maybe be done by infusing ice-cold saline using intravenous (IV) approach.

Although the invention has been described fully above, a number of variations and alterations could be made within the scope of the present invention. For example, in alternative embodiments, steps in the various described methods may be carried out in different orders or skipped altogether, and in other embodiments, additional optional steps may be added or one or more steps may be altered. Therefore, the foregoing description of exemplary embodiments should not be interpreted to limit the scope of the invention described by the following claims.

What is claimed is:

1. A method for removing blood clots from a patient's intracranial space using ultrasound energy, the method comprising:
forming at least one aperture in the patient's skull;
advancing an ultrasound device having a visualization feature through the aperture to a target location within the intracranial space;
transmitting ultrasound energy at frequencies between 1 KHz and 20 MHz from the ultrasound device; and
removing blood clots outside the patient's head.

2. The method of claim 1, wherein the aperture in the skull is selected from the group consisting of: a craniotomy, a burr hole or a twist hole.

3. The method of claim 1, further include removing one of the following; brain tissue, tumor tissue, cerebrospinal fluid, blood, blood clots or combination of all.

4. The method of claim 1, wherein the ultrasound device is placed through an introducer.

5. The method of claim 1, wherein the ultrasound device is placed through a frameless trajectory guide.

6. The method of claim 1, wherein a visualization feature recognizes blood clots in the intracranial space.

7. The method of claim 1, wherein the visualization feature is separate from the ultrasound device.

8. The method of claim 1, wherein the visualization feature comprises an electronic camera positioned on the distal end of the ultrasound device.

9. The method of claim 1, wherein the visualization feature comprises a diagnostic ultrasound visualization component located on the distal end of the ultrasound device.

10. The method of claim 1, wherein the visualization feature comprises an OCT visualization component located on the distal end of the ultrasound device.

11. The method of claim 1, further including repositioning the ultrasound device within the intracranial space.

12. The method of claim 11, wherein repositioning of the ultrasound device is achieved using a deflection, actuation, angulation or pre-shaped telescopic member, or a combination thereof.

13. The method of claim 11, wherein repositioning the ultrasound device comprises advancing the ultrasound device into the one of the following: into the patient's epidural space, into a ventricle of the patient's brain, into an intracerebral space of the patient's brain, or underneath the dura mater of the patient's brain.

14. The method of claim 1, wherein advancing the ultrasound device comprises advancing a device selected from the group consisting of an ultrasound transducer, an ultrasound catheter with a proximal transducer, a transducer-tipped ultrasound catheter, and an ultrasound catheter having multiple transducers located on the distal portion of the catheter.

15. The method of claim 1, further comprising delivering at least one pharmacologic agent to the patient.

16. The method of claim 15, wherein the agent is selected from a group consisting of blood clot reducing agents such as (i) tissue plasminogen activator, tPA, BB-10153, rTPA, Urokinease, Streptokinase, Alteplase and Desmoteplase, (ii) antiplatelet agents such as aspirin, Clopidorgel and Ticclopidine, and (iii) GIIb/IIIa inhibitors, such as Abciximab, Tirofiban and Eptifibatide.

17. The method of claim 1, further comprising delivering microbubbles or nanobubbles to the patient.

18. The method of claim 1, further including delivering irrigation medium through the ultrasound device while transmitting ultrasound energy.

19. The method of claim 1, wherein removal of blood clots to the outside of the patient's head is accomplish using aspiration.

20. A method for removing blood clots from a patient's intracranial space caused by hemorrhage, comprising the step of advancing an ultrasound device into the patient's head, identifying blood clots with a visualization feature, transmitting ultrasound energy at frequencies between 1 KHz and 20 MHz, and removing blood clots outside the head.

21. A method for removing tissue from a patient's intracranial space comprising the step of advancing an ultrasound device into the patient's head, identifying tissue to be removed using a visualization feature, transmitting ultrasound energy at frequencies between 1 KHz and 20 MHz, and removing tissue outside the head.

22. The method of claim 21, wherein the ultrasound device has a deflectable feature that reaches and removes tissue located away from the aperture.

23. A method for removing tissue from a patient's intracranial space comprising the step of advancing a visualization device into the patient's head to identify tissue to be removed, placing an ultrasound device into the treatment area, transmitting ultrasound energy at frequencies between 1 KHz and 20 MHz, and removing tissue outside the head.

24. A method for removing blood clots from a patient's intracranial space using a therapeutic apparatus, the method comprising:
forming at least one aperture in the patient's skull;
advancing a therapeutic apparatus having a visualization feature and an ultrasound feature through the aperture to a target location within the intracranial space;
identifying blood clots;
transmitting ultrasound energy from the ultrasound device; and
removing blood clots outside the patient's head.

25. The method of claim 24, wherein the therapeutic apparatus has an ultrasound device and a visualization device that can be advanced to the intracranial space separately.
